(19) United States
(12) Patent Application Publication
(10) Pub. No.: US 2022/0193420 A1
(43) Pub. Date: Jun. 23, 2022

(54) Defibrillation Through Synchronous Multisite Pacing

(71) Applicant: Texas Heart Institute, Houston, TX (US)

(72) Inventors: Mehdi Razavi, Houston, TX (US); Mathews Medayil John, Houston, TX (US); Allison Davis Post, Houston, TX (US)

(21) Appl. No.: 17/498,402

(22) Filed: Oct. 11, 2021

Related U.S. Application Data
(60) Provisional application No. 63/126,754, filed on Dec. 17, 2020.

(51) Int. Cl. A61N 1/368; A61N 1/05

(52) U.S. Cl. CPC A61N 1/36842; A61N 1/3686; A61N 1/0565

(57) ABSTRACT

An implantable device comprises a plurality of electrode pairs, a sensing unit, and a pacing unit. The electrode pairs comprise a first electrode pair. The first electrode pair is configured to implant at or near a first location of a heart. The sensing unit is configured to sense electrical activity in the heart, determine that the electrical activity indicates an abnormal rhythm, determine a feature of the electrical activity, and select the first electrode pair from the electrode pairs based on the feature. The pacing unit is configured to cause, in response to the abnormal rhythm and the feature, the first electrode pair to provide a first electrical pulse at a first time.

### CROSS-REFERENCE TO RELATED APPLICATIONS

This claims priority to U.S. Prov. Patent App. No. 63/126,754 filed on Dec. 17, 2020, which is incorporated by reference.

### STATEMENT REGARDING FEDERALLY SPONSORED RESEARCH OR DEVELOPMENT

Not applicable.

### BACKGROUND

SCD is death caused by a sudden, unexpected loss of heart function. SCD is the largest cause of natural death in the United States. VF and VT are common causes of SCD. VF is an abnormal heart rhythm in which the ventricles quiver instead of pumping normally. VT is a regular, fast heart rate due to abnormal electrical activity in the ventricles. VF and VT can be treated with ablative and antiarrhythmic therapies. Ablative therapies consist of cauterizing a small portion of the heart responsible for the VF or VT. Antiarrhythmic therapies consist mostly of drugs that block actions on sodium, potassium, or calcium or that block adrenergic receptors. However, cardiac defibrillation remains the most reliable way to combat VF and VT and restore the normal sinus rhythm in the acute setting.

Similarly, AF can cause stroke and other serious clinical consequences. AF is an abnormal and unsynchronized quivering of the atrium. AF can also be treated with ablative and antiarrhythmic therapies. However, again, cardiac defibrillation remains the most reliable treatment.

### SUMMARY OF THE DISCLOSURE

In a first embodiment, an implantable device comprises a plurality of electrode pairs, a sensing unit, and a pacing unit. The plurality of electrode pairs comprises a first electrode pair. The first electrode pair is configured to implant at or near a first location of a heart. The sensing unit is configured to sense electrical activity in the heart, determine that the electrical activity indicates an abnormal rhythm, determine a feature of the electrical activity, and select the first electrode pair from the electrode pairs based on the feature. The pacing unit is configured to cause, in response to the abnormal rhythm and the feature, the first electrode pair to provide a first electrical pulse at a first time.

In implementations of the first embodiment, the electrode pairs comprise a second electrode pair, the second electrode pair is configured to implant at a second location, and the pacing unit is further configured to cause the second electrode pair to provide a second electrical pulse at a second time after the first time. The electrode pairs comprise a third electrode pair, the third electrode pair is configured to implant at a third location, the second location is between the first location and the third location, and the pacing unit is further configured to cause the third electrode pair to provide a third electrical pulse at a third time after the second time. The pacing unit is further configured to cause the second electrode pair to provide a fourth electrical pulse at a fourth time after the third time. The pacing unit is further configured to cause the first electrode pair to provide a fifth electrical pulse at a fifth time after the fourth time. A pulse width (PW) of the first electrical pulse, the second electrical pulse, the third electrical pulse, the fourth electrical pulse, and the fifth electrical pulse is about at least 40 microseconds (μs). A time off between the first electrical pulse and the second electrical pulse, the second electrical pulse and the third electrical pulse, the third electrical pulse and the fourth electrical pulse, and the fourth electrical pulse and the fifth electrical pulse is about at least 1 millisecond (ms). A cycle length (CL) between the first electrical pulse and the second electrical pulse, the second electrical pulse and the third electrical pulse, the third electrical pulse and the fourth electrical pulse, and the fourth electrical pulse and the fifth electrical pulse is at least 1.04 milliseconds (ms). An energy of the first electrical pulse is configured to provide a desirable therapy outcome and configured to be under a pain threshold or a perceptible threshold. The energy is about no more than 2 millijoules (mJ). The feature is a location of earliest activation, a dominant frequency, an organizational index, a timing between local activation, a signal morphology, or a patient-specific feature. The electrode pairs are configured to be implanted only in an atrium of the heart. The electrode pairs are configured to be implanted only in a ventricle of the heart.

In a second embodiment, a method comprises sensing electrical activity in a heart, determining that the electrical activity indicates an abnormal rhythm, determining a feature of the electrical activity, selecting a first electrode pair from a plurality of electrode pairs based on the feature, and causing, in response to the abnormal rhythm and the feature, the first electrode pair to provide a first electrical pulse at a first time and at or near a first location of the heart.

In implementations of the second embodiment, the method further comprises causing a second electrode pair to provide a second electrical pulse at a second time and at a second location of the heart, wherein the second time is after the first time. The method further comprises causing a third electrode pair to provide a third electrical pulse at a third time and at a third location of the heart, wherein the third time is after the second time, and wherein the second location is between the first location and the third location. The method further comprises causing the second electrode pair to provide a fourth electrical pulse at a fourth time after the third time. The method further comprises causing the first electrode pair to provide a fifth electrical pulse at a fifth time after the fourth time.

In a third embodiment, a computer program product comprises instructions that are stored on a computer-readable medium and that, when executed by a processor, cause an implantable device to sense electrical activity in a heart, determine that the electrical activity indicates an abnormal rhythm, determine a feature of the electrical activity, select a first electrode pair from a plurality of electrode pairs based on the feature, and cause, in response to the abnormal rhythm and the feature, the first electrode pair to provide a first electrical pulse at a first time and at or near a first location of the heart.

In implementations of the third embodiment, the feature is a location of earliest activation, a dominant frequency, an organizational index, a timing between local activation, a signal morphology, or a patient-specific feature.

Any of the above embodiments may be combined with any of the other above embodiments to create a new embodiment. These and other features will be more clearly understood from the following detailed description taken in conjunction with the accompanying drawings and claims.

### BRIEF DESCRIPTION OF THE DRAWINGS

For a detailed description of various exemplary embodiments, reference will now be made to the accompanying drawings in which:

FIG. 1 is a schematic diagram of a patient.

FIG. 2A is a schematic diagram 200 showing wireless endocardial placement of the electrode pairs in FIG. 1.

FIG. 2B is a schematic diagram 210 showing wireless epicardial placement of the electrode pairs in FIG. 1.

FIG. 2C is a schematic diagram 220 showing wired endocardial placement of the electrode pairs in FIG. 1.

FIG. 2D is a schematic diagram 230 showing wired epicardial placement of the electrode pairs in FIG. 1.

FIG. 3 is a schematic diagram of the electronic unit in FIG. 1.

FIG. 4 is a method of multisite pacing for defibrillation.

FIG. 5 is a graph illustrating electrode pair activation.

FIG. 6 is another graph illustrating electrode pair activation.

### DETAILED DESCRIPTION OF THE DISCLOSED EXEMPLARY EMBODIMENTS

The following discussion is directed to various embodiments. However, one of ordinary skill in the art will understand that the examples disclosed herein have broad application, and that the discussion of any embodiment is meant only to be exemplary of that embodiment, and not intended to suggest that the scope of the disclosure, including the claims, is limited to that embodiment.

The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection of the two devices, or through an indirect connection that is established via other devices, components, nodes, and connections. In addition, as used herein, the terms “axial” and “axially” generally mean along or parallel to a given axis (e.g., central axis of a body or a port), while the terms “radial” and “radially” generally mean perpendicular to the given axis. For instance, an axial distance refers to a distance measured along or parallel to the axis, and a radial distance means a distance measured perpendicular to the axis. Further, when used herein (including in the claims) in connection with a stated value, the words “about,” “generally,” “substantially,” “approximately,” and the like mean within a range of plus or minus 10% of the stated value.

The following abbreviations apply:

AF: atrial fibrillation

AHA: American Heart Association

CL: cycle length

CPU: central processing unit

E: electrode pair

ICD: implantable cardioverter-defibrillator

J: joule(s)

mJ: millijoule(s)

ms: millisecond(s)

PS: pacing site

PTSD: post-traumatic stress disorder

PW: pulse width

RAM: random-access memory

ROM: read-only memory

s: second(s)

SCD: sudden cardiac death

VF: ventricular fibrillation

VT: ventricular tachycardia

μJ: microjoule(s)

μs: microsecond(s).

During defibrillation, a high-energy electrical shock is applied to the heart. The supplied energy temporarily blocks the progression of the uncoordinated waveforms that result in VF, VT, and AF. The overall mechanism of defibrillation is still being widely studied. According to AHA guidelines, external defibrillators that produce biphasic waveforms are preferred over monophasic waveforms, and the biphasic waveforms should be 200 J or less for a shock to combat hemodynamically unstable VF and VT. For VF, the shock is the first shock. For VT, the shock follows an anti-tachycardic pacing if the anti-tachycardic pacing fails. Pacing is a relatively low-energy and short-duration electrical stimulation, while shocking is a relatively high-energy and long-duration electrical stimulation.

Use of external defibrillators can be painful, causing an increased incidence of PTSD and depression, which cause a decrease in quality of life. ICDs provide an alternative to external defibrillators. ICDs are transvenously implanted in the heart with the defibrillation coil in the right ventricle. Algorithms embedded inside the ICD provide appropriate therapy after sensing VF, VT, or AF. Although these algorithms have evolved in the past few years, there remains a high incidence of inappropriate shocks. In addition, the pain and uncertain timing of the shocks results in a decrease in quality of life.

Due to the pain associated with defibrillation, current research seeks to provide a defibrillation method using pacing algorithms that provide energies far below the pain threshold of 1-2 J. While many attempts have been made, there are very few successful clinical applications of this approach. There is therefore a desire to provide a defibrillation method that is both painless and effective.

Disclosed herein are embodiments for defibrillation through synchronous multisite pacing. Synchronous means that the pacing is coordinated in time. Multisite means that the pacing occurs through electrode pairs located at different sites of the heart. However, the sites may be confined to one or two atria or ventricles. The pacing may begin at a location of earliest activation in the heart. The pacing may then extend in a first direction and return in a second direction opposite the first direction. Because the pacing is multisite, it may use a lower energy, which could be far below the pain threshold. The synchronous multisite pacing attempts to recreate a sinus rhythm of the heart and thus resynchronize movement of myocardial tissue. This method of synchronous multisite pacing has been successful in terminating AF in at least three large animal studies. While testing may be limited to the atria due to in vivo testing restrictions, defibrillation through synchronous multisite pacing also applies to terminating VF or other arrhythmias in the ventricle. The embodiments may be applied using any multi-electrode device or a combination of multi-electrode devices. The devices may be either endocardially or epicardially placed and may be either temporarily or chronically implanted or applied. Electrode pairs in the devices may be used for both sensing and pacing. Similar techniques may also apply to deep brain stimulations where multiple lobes of the brain or multiple areas in a single lobe are synchronously activated or to synchronous stimulation of muscle throughout the body.

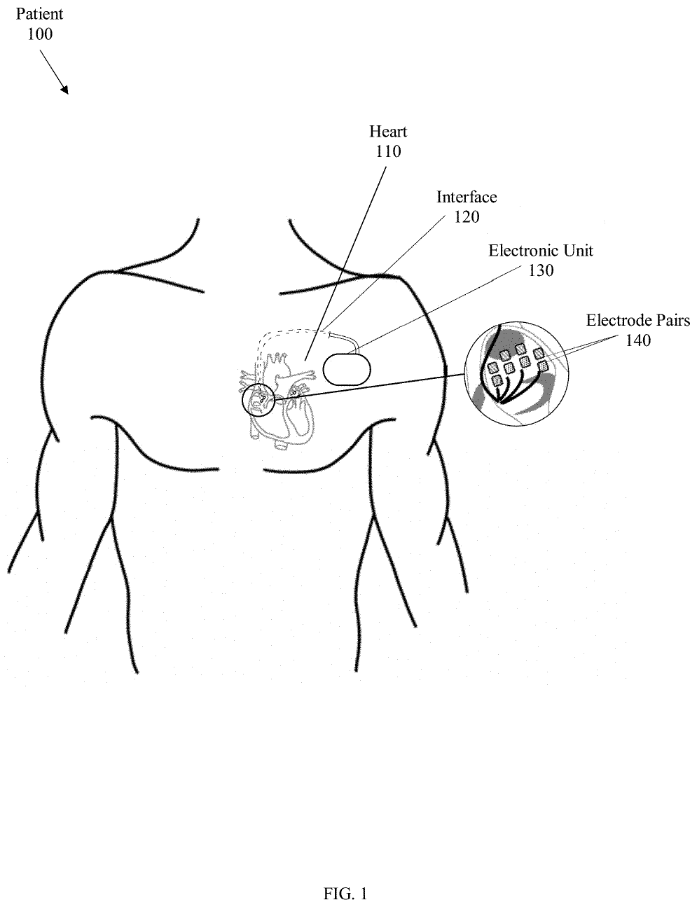

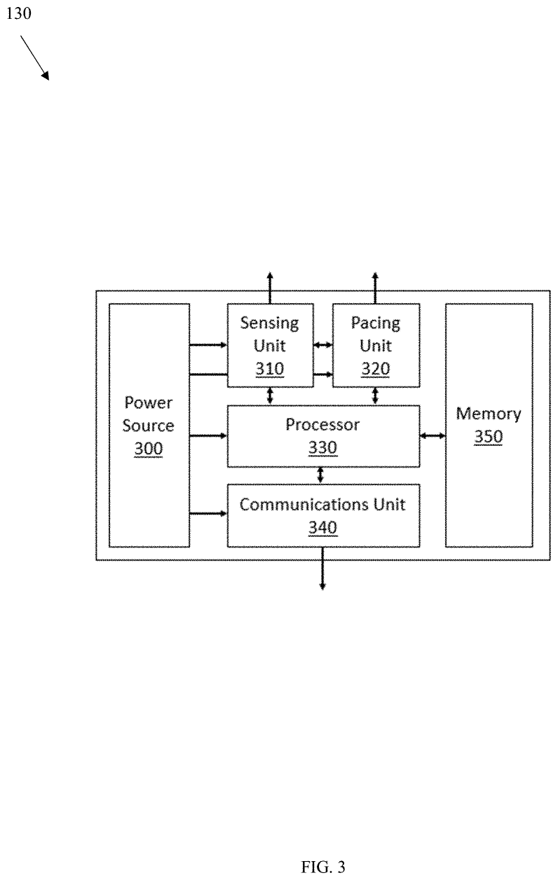

FIG. 1 is a schematic diagram of a patient 100. The patient 100 has a heart 110 and an ICD. The ICD be more generally referred to as an implantable device and comprises an interface 120, an electronic unit 130, and electrode pairs 140. The interface 120 may be wired or wireless. The electronic unit 130 is shown in FIG. 3.

The electrode pairs 140 are implanted within the heart 110 and thus internal. For instance, the electrode pairs 140 are implanted on the atrium. Alternatively, some or all of the electrode pairs 140 are external to the patient 100. Though four electrode pairs 140 are shown, the ICD may have any number of electrode pairs 140. For instance, the ICD may have 2-50 electrode pairs 140. Each electrode pair 140 may comprise two unique electrodes, or each electrode pair 140 may comprise one unique electrode that shares a common indifferent electrode that is shared with the other unique electrodes. The indifferent electrode may be on the heart 110 or in another part of the patient 100. Alternatively, some or all of the electrode pairs 140 have a first electrode active electrode on the heart 110 and a second ground electrode outside the patient 100.

A professional may implant the electrode pairs 140 in strategic locations. For instance, at least one electrode pair 140 may be at or near a location of earliest activation, which is described below. That location may be on or near the septum of the heart 110. In this context, “near” may mean close enough to provide electrical stimulation. The professional may implant additional electrode pairs 140 where VF, VT, or AF often occurs or originates. For instance, AF often originates near the left atrial back wall. Finally, the professional may implant the electrode pairs 140 based on a size and a shape of the electrode pairs 140 and the greater ICD, the proper functioning of the heart 110 without interference from the electrode pairs 140, power conservation of the electronic unit 130, therapy optimization, anatomy of the patient 100, pathologies of the patient 100, and other factors.

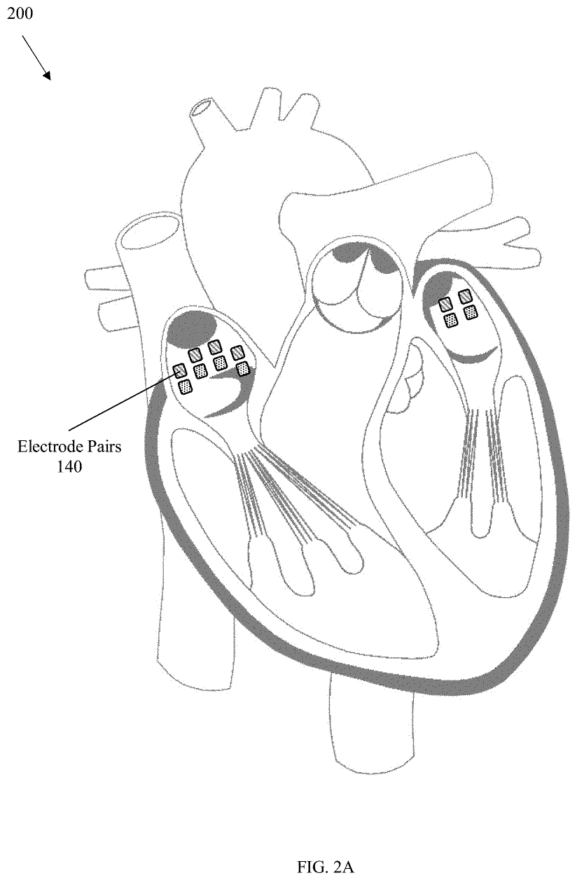

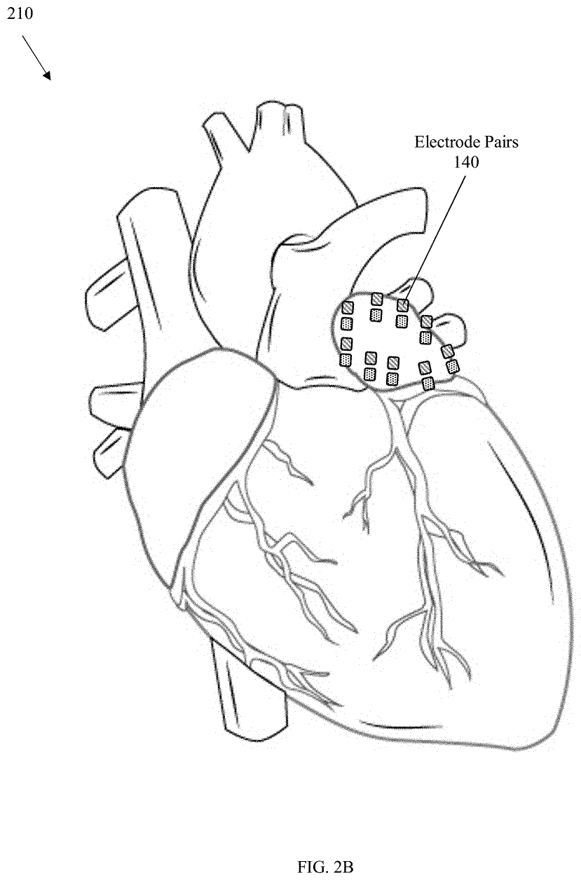

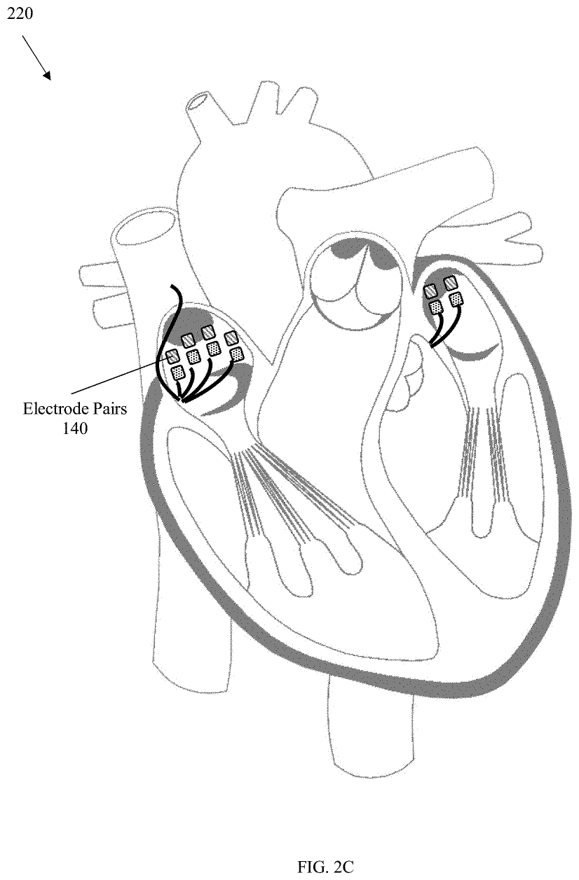

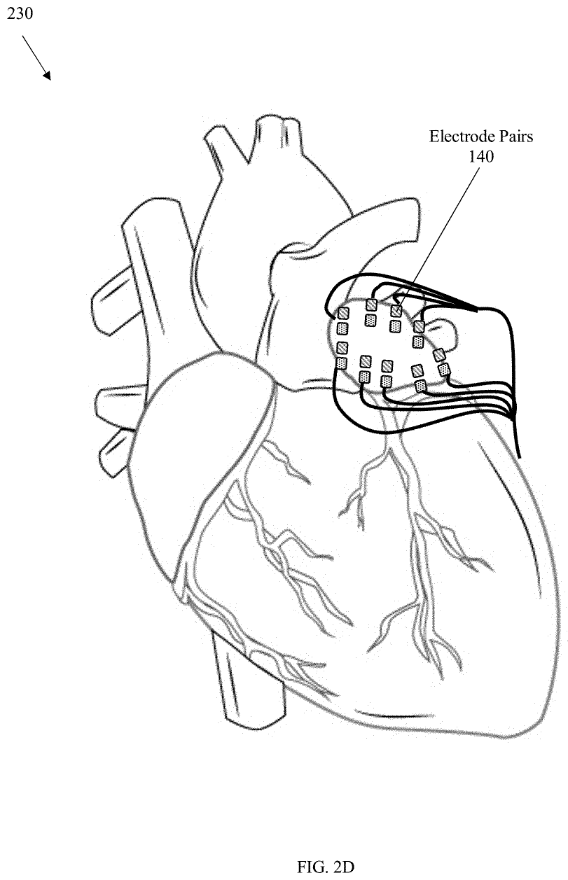

FIG. 2A is a schematic diagram 200 showing wireless endocardial placement of the electrode pairs 140 in FIG. 1. FIG. 2B is a schematic diagram 210 showing wireless epicardial placement of the electrode pairs 140 in FIG. 1. FIG. 2C is a schematic diagram 220 showing wired endocardial placement of the electrode pairs 140 in FIG. 1. FIG. 2D is a schematic diagram 230 showing wired epicardial placement of the electrode pairs 140 in FIG. 1. Though the configurations of the electrode pairs 140 shown in the schematic diagrams 200, 210, 220, 230 are separated, those configurations may be combined in any suitable manner.

FIG. 3 is a schematic diagram of the electronic unit 130 in FIG. 1. The electronic unit 130 comprises a power source 300, a sensing unit 310, a pacing unit 320, a processor 330, a communications unit 340, and a memory 350. The power source 300 is a battery or another device that stores and emits electric current to provide power to the sensing unit 310, the pacing unit 320, the processor 330, and the communications unit 340.

The sensing unit 310 comprises electronic circuitry that can filter, amplify, and digitize electrical signals. The sensing unit 310 uses the electrode pairs 140 to sense the function of the heart 110. That function includes undesirable events such as VF, VT, and AF.

The pacing unit 320 comprises electronic circuitry that can provide electrical pulses of programmable amplitude, PW, and frequency. The pacing unit 320 uses the electrode pairs 140 to provide electrical current to the heart 110 in order to keep or cause a desirable therapy outcome such as defibrillation or a desirable rhythm.

The sensing unit 310 and the pacing unit 320 may be separate processors, part of the processor 330, or part of instructions stored in the memory 350 and executed by the processor 330. The instructions may be in the form of a computer program product. The sensing unit 310 and pacing unit 320 may include a switching mechanism to switch between different electrode pairs 140 to sense and pace from different electrode pairs 140 at different times.

The processor 330 is a microcontroller, a microprocessor, a CPU, or another device that executes the instructions to implement the embodiments. The communications unit 340 uses the interface 120 to communicate with an external device such as a monitor to view data from the electronic unit 130 or a computer to analyze that data or program the electronic unit 130. The memory 350 is a RAM, a ROM, or another computer-readable medium that store stores the instructions.

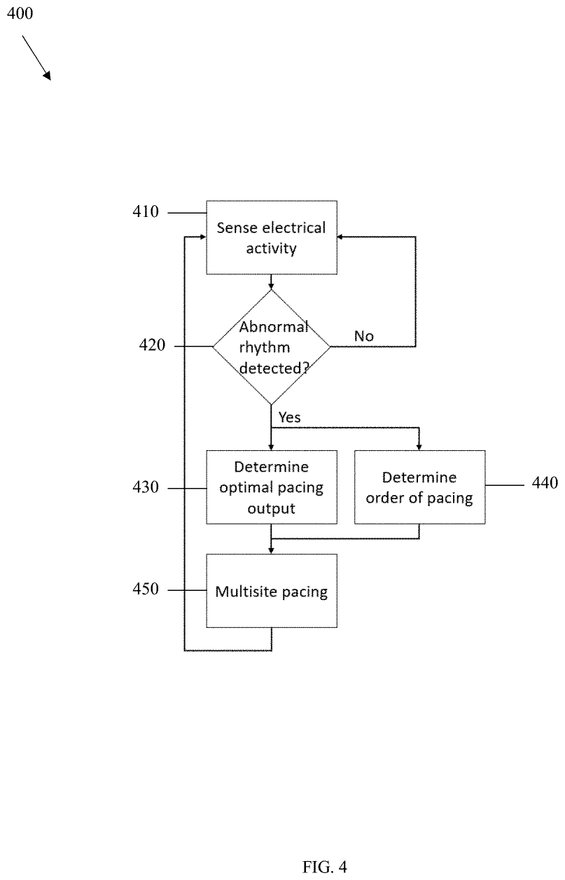

FIG. 4 is a method 400 of multisite pacing for defibrillation. At step 410, the sensing unit 310 senses electrical activity in the heart 110 using the electrode pairs 140. At decision 420, the sensing unit 310 determines whether an abnormal event such as an abnormal rhythm is detected in the electrical activity at step 410. If not, then the method 400 returns to step 410. If so, then the method 400 proceeds to steps 430 and 440. At step 430, the pacing unit 320 determines an optimal pacing output. For instance, the optimal pacing output is an energy of an electrical current from the electrode pairs 140 to the heart 110. The energy provides a desirable outcome, for instance, defibrillation, while also being under a pain threshold or a perceptible threshold. For instance, the energy is no more than 2 mJ. At step 440, the pacing unit 320 determines an order of pacing. For instance, the order of pacing means that a first electrode pair 140 provides an output such as an electrical pulse at a first time, a second electrode pair 140 provides an output at a second time, and so on. The order of pacing is described further below. Finally, at step 450, the pacing unit 320 performs multisite pacing to the heart 110 through the electrode pairs 140 and based on the determinations at steps 430 and 440. After step 450, the method 400 returns to step 410.

The electronic unit 130 may perform a variety of functions. In a first embodiment, the pacing unit 320 begins the order of pacing through an initial electrode pair 140 at or nearest to a location of earliest activation in tissue of the heart 110. In continuous cycles, the heart 110 electrically stimulates, contracts, and pumps. The location of earliest activation is where the heart 110 begins that process in each cycle, in other words, where the first electrical stimulation occurs. The location of earliest activation may be different for different patients. The sensing unit 310 determines the location of earliest activation based on analysis of signals it obtains from the heart 110 and through the electrode pairs 140.

In a second embodiment, the sensing unit 310 determines features of electrical signals sensed from the heart 110 and through the electrode pairs 140. While the features may include the location of earliest activation as described above, the features may also include a dominant frequency, an organizational index, timing between local activation, a signal morphology, or features specific to the patient 100. The dominant frequency is the fastest rate at which the myocardial tissue is being excited. The organizational index is a measure of an amount of tissue being activated at the dominant frequency. Signal morphologies could include a slope, an amplitude, or a frequency of the signal and may be derived from a Fourier transform. Based on those features, the sensing unit 310 determines a first electrode pair 140 for pacing, and the pacing unit 320 begins the order of pacing at the first electrode pair 140.

In a third embodiment, the pacing unit 320 continues pacing through subsequent electrode pairs 140 at subsequent locations after predetermined time delays. The predetermined time delays may be based on proximities of the subsequent electrode pairs 140 to the initial electrode pair 140 or sensed information from the sensing unit 310. Pacing at multiple locations after time delays may simulate a contiguous pacing, or shock wave propagating through the atrium of the heart 110.

In a fourth embodiment, the pacing unit 320 paces through the electrode pairs 140 in a first direction. The pacing unit 320 then paces through the electrode pairs 140 in a second direction that is opposite the first direction. For instance, the pacing unit 320 paces in the order E1, E2, E3, E2, E1, where E1 is a first electrode pair 140, E2 is a second electrode pair 140, and E3 is a third electrode pair 140. The pacing unit 320 continues that pacing order until the sensing unit 310 determines that an undesirable condition such as an arrhythmia has ceased. The predetermined time delays between pacing at each electrode pair 140 may be different between the first direction and the second direction.

In a fifth embodiment, the pacing unit 320 paces through the electrode pairs 140 only in a first direction. For instance, the pacing unit 320 paces in the order E1, E2, E3, E1, E2, E3, and so on. The pacing unit 320 continues that pacing order until the sensing unit 310 determines that an undesirable condition has ceased.

In a sixth embodiment, the sensing unit 310 monitors the heart 110 through the electrode pairs 140. Based on that monitoring, the sensing unit 310 determines an overall health of the myocardium of the heart 110.

The pacing unit 320 implements pacing parameters through the electrode pairs 140. The pacing parameters comprise a PW, a time off, and a CL. The PW is the amount of time that the electrode pairs 140 emit an electrical pulse. The PW may be about 40 μs-1.0 s, for instance, at least 40 μs or about 100 ms. The time off is the amount of time between the end of a first electrical pulse and the beginning of a second electrical pulse. The time off may be about 1-200 ms, for instance, at least 1 ms, and may vary between sets of electrical pulses. The CL is the amount of time between the middle of a first electrical pulse and the middle of a second electrical pulse. The CL is equal to the sum of the PW and the time off between two electrical pulses. The CL may be about 1.04 ms-1.2 s, for instance, at least 1.04 ms or about 150-300 ms.

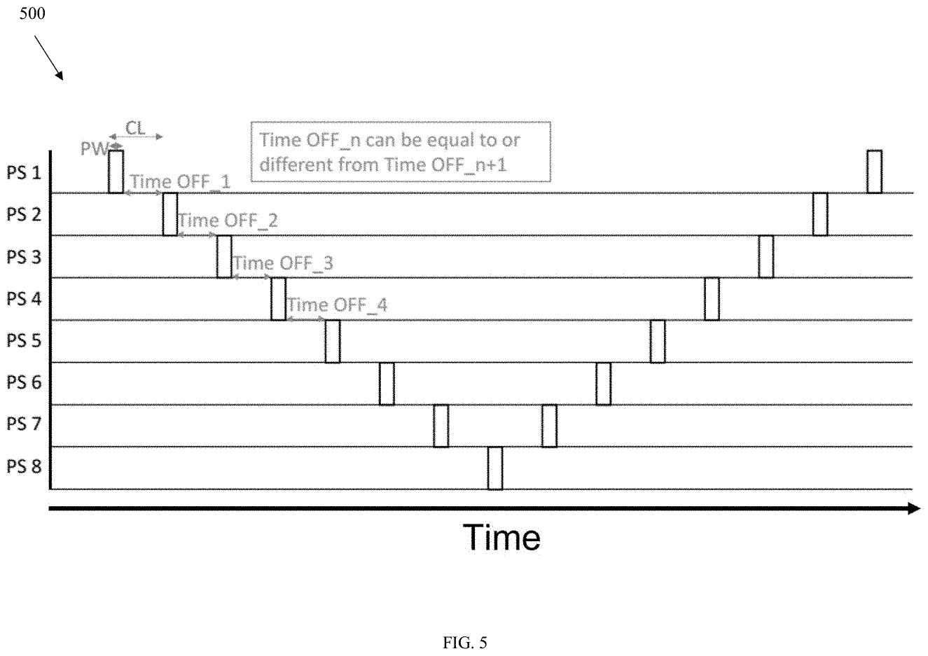

FIG. 5 is a graph 500 illustrating electrode pair activation. The graph 500 corresponds to the third embodiment described above. Moving from left to right in time, the graph 500 shows that a first electrode pair PS 1 provides a first pulse, a second electrode pair PS 2 provides a second pulse, a third electrode pair PS 3 provides a third pulse, a fourth electrode pair PS 4 provides a fourth pulse, a fifth electrode pair PS 5 provides a fifth pulse, a sixth electrode pair PS 6 provides a sixth pulse, a seventh electrode pair PS 7 provides a seventh pulse, and an eighth electrode pair PS 8 provides an eighth pulse. Then PS 7 provides a ninth pulse, PS 6 provides a tenth pulse, PS 5 provides an eleventh pulse, PS 4 provides a twelfth pulse, PS 3 provides a thirteenth pulse, PS 2 provide a fourteenth pulse, and PS 1 provides a fifteenth pulse. The graph 500 demonstrates the PW, the time off, and the CL. The time off between the first pulse and the second pulse is denoted as time off 1, the time off between the second pulse and the third pulse is denoted as time off 2, the time off between the third pulse and the fourth pulse is denoted as time off 3, and the time off between the fourth pulse and the fifth pulse is denoted as time off 4.

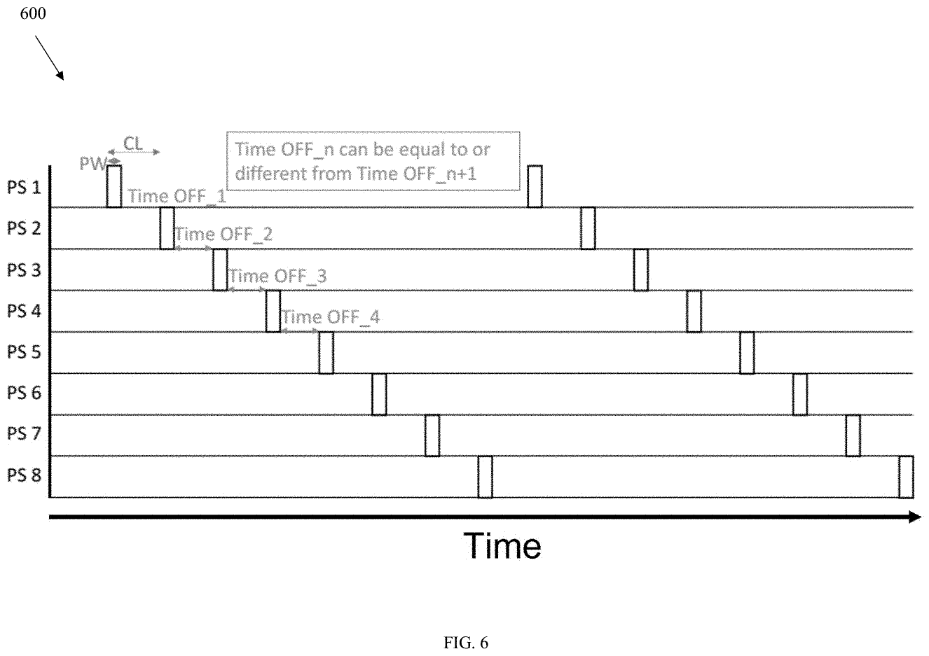

FIG. 6 is another graph 600 illustrating electrode pair activation. The graph 600 corresponds to the fourth embodiment described above. Moving from left to right in time, the graph 600 shows that a first electrode pair PS 1 provides a first pulse, a second electrode pair PS 2 provides a second pulse, a third electrode pair PS 3 provides a third pulse, a fourth electrode pair PS 4 provides a fourth pulse, a fifth electrode pair PS 5 provides a fifth pulse, a sixth electrode pair PS 6 provides a sixth pulse, a seventh electrode pair PS 7 provides a seventh pulse, and an eighth electrode pair PS 8 provides an eighth pulse. Then PS 1 provides a ninth pulse, PS 2 provides a tenth pulse, PS 3 provides an eleventh pulse, PS 4 provides a twelfth pulse, PS 5 provides a thirteenth pulse, PS 6 provide a fourteenth pulse, PS 7 provides a fifteenth pulse, and PS 8 provides a sixteenth pulse. The graph 600 demonstrates the PW, the time off, and the CL. The time off between the first pulse and the second pulse is denoted as time off 1, the time off between the second pulse and the third pulse is denoted as time off 2, the time off between the third pulse and the fourth pulse is denoted as time off 3, and the time off between the fourth pulse and the fifth pulse is denoted as time off 4.

While exemplary embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. An implantable device comprising:
a plurality of electrode pairs comprising a first electrode pair, wherein the first electrode pair is configured to implant at or near a first location of a heart;
a sensing unit configured to:
sense electrical activity in the heart,
determine that the electrical activity indicates an abnormal rhythm,
determine a feature of the electrical activity, and
select the first electrode pair from the electrode pairs based on the feature; and
a pacing unit configured to cause, in response to the abnormal rhythm and the feature, the first electrode pair to provide a first electrical pulse at a first time.

2. The implantable device of claim 1, wherein the electrode pairs comprise a second electrode pair, wherein the second electrode pair is configured to implant at a second location, and wherein the pacing unit is further configured to cause the second electrode pair to provide a second electrical pulse at a second time after the first time.

3. The implantable device of claim 2, wherein the electrode pairs comprise a third electrode pair, wherein the third electrode pair is configured to implant at a third location, wherein the second location is between the first location and the third location, and wherein the pacing unit is further configured to cause the third electrode pair to provide a third electrical pulse at a third time after the second time.

4. The implantable device of claim 3, wherein the pacing unit is further configured to cause the second electrode pair to provide a fourth electrical pulse at a fourth time after the third time.

5. The implantable device of claim 4, wherein the pacing unit is further configured to cause the first electrode pair to provide a fifth electrical pulse at a fifth time after the fourth time.

6. The implantable device of claim 5, wherein a pulse width (PW) of the first electrical pulse, the second electrical pulse, the third electrical pulse, the fourth electrical pulse, and the fifth electrical pulse is at least 40 microseconds (μs).

7. The implantable device of claim 5, wherein a time off between the first electrical pulse and the second electrical pulse, the second electrical pulse and the third electrical pulse, the third electrical pulse and the fourth electrical pulse, and the fourth electrical pulse and the fifth electrical pulse is at least 1 millisecond (ms).

8. The implantable device of claim 5, wherein a cycle length (CL) between the first electrical pulse and the second electrical pulse, the second electrical pulse and the third electrical pulse, the third electrical pulse and the fourth electrical pulse, and the fourth electrical pulse and the fifth electrical pulse is at least 1.04 milliseconds (ms).

9. The implantable device of claim 1, wherein an energy of the first electrical pulse is configured to provide a desirable therapy outcome and configured to be under a pain threshold or a perceptible threshold.

10. The implantable device of claim 9, wherein the energy is no more than 2 millijoules (mJ).

11. The implantable device of claim 1, wherein the feature is a location of earliest activation, a dominant frequency, an organizational index, a timing between local activation, a signal morphology, or a patient-specific feature.

12. The implantable device of claim 1, wherein the electrode pairs are configured to be implanted only in an atrium of the heart.

13. The implantable device of claim 1, wherein the electrode pairs are configured to be implanted only in a ventricle of the heart.

14. A method comprising:
sensing electrical activity in a heart;
determining that the electrical activity indicates an abnormal rhythm;
determining a feature of the electrical activity;
selecting a first electrode pair from a plurality of electrode pairs based on the feature; and
causing, in response to the abnormal rhythm and the feature, the first electrode pair to provide a first electrical pulse at a first time and at or near a first location of the heart.

15. The method of claim 14, further comprising causing a second electrode pair to provide a second electrical pulse at a second time and at a second location of the heart, wherein the second time is after the first time.

16. The method of claim 15, further comprising causing a third electrode pair to provide a third electrical pulse at a third time and at a third location of the heart, wherein the third time is after the second time, and wherein the second location is between the first location and the third location.

17. The method of claim 16, further comprising causing the second electrode pair to provide a fourth electrical pulse at a fourth time after the third time.

18. The method of claim 17, further comprising causing the first electrode pair to provide a fifth electrical pulse at a fifth time after the fourth time.

19. A computer program product comprising instructions that are stored on a computer-readable medium and that, when executed by a processor, cause an implantable device to:
sense electrical activity in a heart;
determine that the electrical activity indicates an abnormal rhythm;
determine a feature of the electrical activity;
select a first electrode pair from a plurality of electrode pairs based on the feature; and
cause, in response to the abnormal rhythm and the feature, the first electrode pair to provide a first electrical pulse at a first time and at or near a first location of the heart.

20. The computer program product of claim 19, wherein the feature is a location of earliest activation, a dominant frequency, an organizational index, a timing between local activation, a signal morphology, or a patient-specific feature.
